(19) United States
(12) Patent Application Publication
(10) Pub. No.: US 2022/0257189 A1
(43) Pub. Date: Aug. 18, 2022

(54) ADHESIVE BANDAGES AND METHODS FOR THEIR USE AND MANUFACTURE

(71) Applicant: Meghan Sharkus, Minneapolis, MN (US)

(72) Inventor: Meghan Sharkus, Minneapolis, MN (US)

(21) Appl. No.: 17/628,188

(22) PCT Filed: Jul. 17, 2020
(86) PCT No.: PCT/US20/42602

§ 371 Date: Jan. 18, 2022

Related U.S. Application Data
(60) Provisional application No. 62/875,726, filed on Jul. 18, 2019.

(51) Int. Cl. A61B 5/00; A61B 5/145

(52) U.S. Cl. CPC A61B 5/6833; A61B 2560/0412; A61B 5/14532

(57) ABSTRACT

The present disclosure relates to an adhesive bandage, as well as methods of using and manufacturing the same. A benefit to the adhesive bandage can be providing the use of a single adhesive bandage for adhering a subcutaneous sensor to the skin of a patient in an efficient and hygienic manner.

### TECHNICAL FIELD

The present disclosure relates to adhesive bandages, as well as methods of using and manufacturing the same. A benefit to the adhesive bandages can be providing the use of a single adhesive bandage for adhering a subcutaneous sensor to the skin of a subject in an efficient and hygienic manner.

### BACKGROUND

Adhesive bandages are widely used to attach subcutaneous sensors in place to the skin of medical patients in need of regular sensor monitoring. Adhesive bandages are generally expected to provide a breathable, protective hygienic covering for the skin. Adhesive bandages used to attach sensors to the skin are also expected to firmly bind a portion of the sensor positioned above the skin surface. Various approaches are used for the attachment of subcutaneous sensors to the skin using adhesive bandages. For example, for continuous glucose monitoring, a subcutaneous glucose sensor can be attached initially to the skin of a patient using an adhesive pad. To further stabilize and protect the sensor and the skin, the sensor can be taped over by one or more adhesive bandages. Further, a sensor transmitter, connected to the sensor, can also be stabilized and covered using one or more adhesive bandages.

There is a need for an adhesive bandage that can attach a subcutaneous sensor to the skin of a subject and allow for any wires, tubes, or transmitters to extend through the material of the bandage. There is a need for an adhesive bandage that can be attached to the skin by the subject or a health care worker in one or two steps and without the need to touch the adhesive layer of the bandage. There is a need for an adhesive bandage that can comfortably attach a sensor to skin for long periods of wear. There is a need for an adhesive bandage that can provide a pleasing, but functional appearance to guide users in the proper application of the adhesive bandage.

### SUMMARY

Embodiments herein are directed to adhesive bandages, methods of manufacturing the same, and methods for the application thereof.

In an aspect, an adhesive bandage comprises a front layer, wherein the front layer includes a nonwoven material and the front layer has an upper surface, a lower surface, and an outer edge; an adhesive layer adhered to at least a portion of the lower surface of the front layer; and a backing layer attached to the adhesive layer. In such an aspect, the front layer and the adhesive layer include at least two separate bandage panels, wherein the at least two separate bandage panels are divided by at least one front slit, and the at least two separate bandage panels include a port front panel and a non-port front panel, wherein the port front panel includes a port opening extending through at least the front layer. In such an aspect, the backing layer includes at least two backing panels, wherein the at least two backing panels include a port back panel and a non-port back panel, wherein the at least two backing panels are separated by at least one back slit; and wherein the at least one back slit is a first overhang distance from the front slit.

In certain embodiments, the front layer has a length of from about 7.5 cm to about 15.2 cm, and a width of from about 7.5 cm to about 12.7 cm. In certain embodiments, the port opening has a length of from about 1.7 cm to about 3.8 cm, and a width of from about 0.7 cm to about 1.8 cm. In certain embodiments, the front layer has a thickness of from about 2 micrometers to about 90 micrometers. In certain embodiments, the front layer and the adhesive layer have a combined thickness of from about 50 micrometers to about 150 micrometers. In certain embodiments, the backing layer has a thickness of from about 50 micrometers to about 150 micrometers, including from about 80 to 120 micrometers. In certain embodiments, the first overhang distance of the at least one back slit from the front slit is from about 0.5 cm to about 1.8 cm.

In various embodiments, the nonwoven material can include polyurethane, polyvinylchloride, polyethylene, polyester, rayon, synthetic rubber, an elastic fabric, or combinations thereof.

In an embodiment, the adhesive layer is formed from a tacky adhesive material. In various embodiments, the tacky adhesive material can include an acrylate resin, a methacrylate resin, an epoxy resin, and combinations thereof.

In various embodiments, the backing layer can be formed from a nonwoven elastic polymer material, a flexible plastic, a wax coated paper material, a silicone treated paper material, or a combination thereof.

In certain embodiments, the port back panel and the non-port back panel are separated by a second back slit, wherein the second back slit is a second overhang distance from the front slit; and wherein the backing layer further comprises a bridging back panel between the first back slit and the second back slit. In certain embodiments, the second overhang distance of the second back slit from the front slit is from about 0.5 cm to about 1.8 cm.

In other embodiments, the adhesive bandage further comprises a display layer, wherein in various embodiments the display layer can include an ink, a dye, a pigment, or a combination thereof. In certain embodiments, the front layer of an adhesive bandage is printed with a decorative design. In certain embodiments, the display layer includes an unresolved puzzle pattern, and the unresolved puzzle pattern is configured to be resolved into a resolved pattern if the at least two separate bandage panels are overlapped by about the first overhang distance.

In certain embodiments, the front layer has a circular or an oval shape. In certain embodiments, the port opening has a circular or an oval shape.

In certain embodiments, the port opening has an inner edge, wherein the adhesive layer does not extend to be coterminous with the outer edge of the front layer. In certain embodiments, the adhesive layer does not extend to be coterminous with the inner edge of the port opening.

A method of adhering a sensor to a skin of a subject in need thereof is disclosed herein. In an embodiment, the method includes placing the sensor into contact with the skin of the subject; providing an adhesive bandage according to embodiments herein; adhering at least a portion of the port front layer into contact with an adhesive portion of the sensor and at least a portion of the port front layer into contact with the skin of the subject; and adhering at least a portion of the non-port front layer into contact with the upper surface of the port front layer and onto the skin of the subject.

In additional embodiments, a method of adhering a sensor to a skin of a subject in need thereof further comprises connecting a sensor transmitter through the port opening to the sensor before, during, or after adhering the port front layer into contact with the sensor and the skin of the subject. In an embodiment, the sensor includes a sensor portion, the adhesive portion, and an exterior portion.

A method of manufacturing an adhesive bandage is disclosed herein. In an embodiment, the method includes providing a pre-bandage material, wherein the pre-bandage material includes a front layer, wherein the front later includes a nonwoven material and the front layer has an upper surface, a lower surface, and an outer edge; an adhesive layer adhered to at least a portion of the lower surface of the front layer; and a backing layer attached to the adhesive layer; and cutting at least one of the front layer and the backing layer of the pre-bandage material to form an adhesive bandage according to embodiments herein.

### BRIEF DESCRIPTION OF THE DRAWINGS

The foregoing summary, as well as the following detailed description of the embodiments, will be better understood when read in conjunction with the attached drawings. For the purpose of illustration, there are shown in the drawings some embodiments, which may be preferable. It should be understood that the embodiments depicted are not limited to the precise details shown. Unless otherwise noted, the drawings are not to scale.

FIG. 1A shows a schematic top view of an embodiment of an adhesive bandage.

FIG. 1B shows a side view of the embodiment of the adhesive bandage in FIG. 1A.

FIG. 2 depicts an embodiment of a method of adhering the adhesive bandage of FIG. 1B to skin, a sensor, and a transmitter.

FIG. 3A shows a schematic depiction of the embodiment of the adhesive bandage from FIG. 1A, having an unresolved ink pattern.

FIG. 3B shows a schematic depiction of the embodiment of the adhesive bandage from FIG. 3A, having a resolved ink pattern.

FIG. 3C shows a side view of the embodiment of the adhesive bandage in FIG. 3B.

### DETAILED DESCRIPTION

Unless otherwise noted, all measurements are in standard metric units.

Unless otherwise noted, all instances of the words “a,” “an,” or “the” can refer to one or more than one of the word that they modify.

Unless otherwise noted, the phrase “at least one of” means one or more than one of an object. For example, “at least one back slit” means one back slit, more than one back slit, or any combination thereof.

Unless otherwise noted, the term “about” refers to ±10% of the non-percentage number that is described, rounded to the nearest whole integer. For example, about 100 mm, would include 90 to 110 mm. Unless otherwise noted, the term “about” refers to ±5% of a percentage number. For example, about 20% would include 15 to 25%. When the term “about” is discussed in terms of a range, then the term refers to the appropriate amount less than the lower limit and more than the upper limit. For example, from about 100 to about 200 mm would include from 90 to 220 mm.

The term “tacky” as used herein refers to an adhesive capable of adhering to skin upon contact under ambient conditions and without additional treatment, such as the addition of chemicals or photocuring.

The term “subject” refers to a patient, a human, or an animal, including mammals.

Unless otherwise noted, dimension measurements, including height, width, length, and thickness, are understood to be averaged measurements.

### Embodiments of Adhesive Bandages

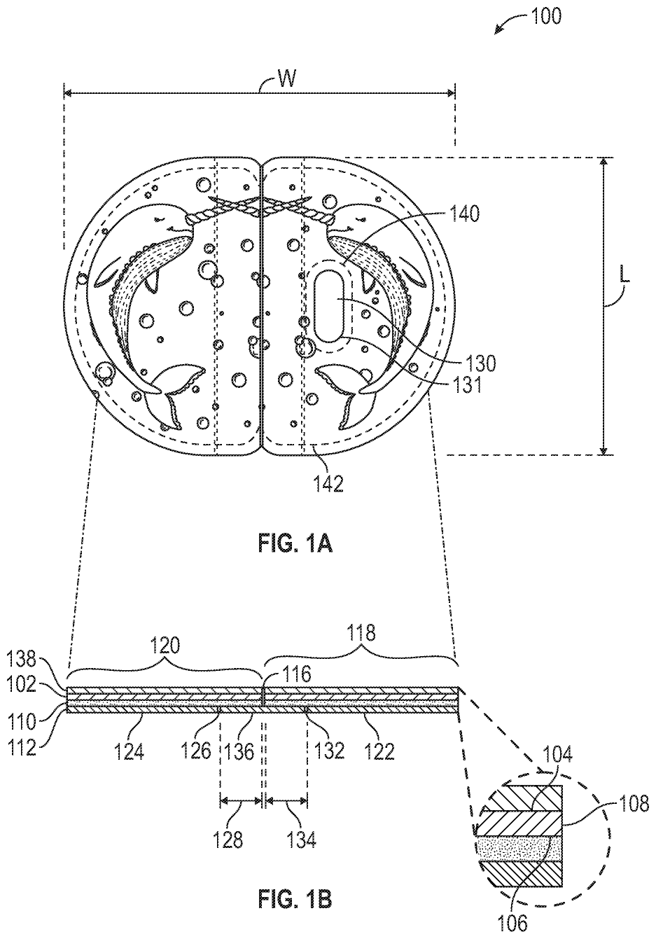

An embodiment of an adhesive bandage as disclosed herein is shown in FIG. 1A with a side view shown in FIG. 1B. The adhesive bandage 100 has a width W and a length L. Referring to FIG. 1B, the adhesive bandage includes a Front Layer 102 having an upper surface 104, a lower surface 106, and an outer edge 108. The adhesive bandage includes an Adhesive Layer 110 adhered to at least a portion of the Front Layer lower surface. The adhesive bandage includes a Backing Layer 112 attached to the Adhesive Layer. The Front Slit 116 divides the Front Layer and the Adhesive Layer into a port front panel 118 and a non-port front panel 120. The Backing Layer includes a port back panel 122 and a non-port back panel 124 separated by a first back slit 126 at first overhang distance 128 from the Front Slit. Referring to FIG. 1A, the port front panel includes port opening 130, extending through at least the Front Layer of the port front panel, and the port opening has an inner edge 131 of the Front Layer.

In certain embodiments, referring to FIG. 1B, the adhesive bandage Backing Layer has second back slit 132 at a second overhang distance 134 from the Front Slit. In such an embodiment, the first back slit and the second back slit form a bridging back panel 136 in the Backing Layer.

In certain embodiments, referring to FIG. 1B, the adhesive bandage has an Ink Layer 138 attached at least a portion of the upper surface of the Front Layer. In certain embodiments, referring to FIG. 1A, the adhesive layer does not extend to the edge of the Front Layer, forming an inner adhesive layer edge 140 and an outer adhesive layer edge 142.

Many types of subcutaneous sensors in current use include a sensor portion, a sensor port, and an adhesive portion. In general, the sensor portion is inserted beneath the skin surface, and provides measurement data. The sensor portion can be connected to a sensor port that extends above the skin surface to transmit the sensor data. A transmitter can be attached to the sensor port to transmit the sensor data. An adhesive portion or pad can be included as part of the sensor assembly to aid in the initial attachment of the sensor to the skin, once the sensor portion is inserted beneath the skin surface.

Conventional over-tape bandages include a port opening and can be applied over the sensor port and attached to the skin of the patient or subject, overlapping the adhesive pad and the skin, with the sensor port or wires extending through the port opening and above the skin surface. A separate sensor transmitter can be connected to the sensor port. One or more additional over-tape bandages can be applied to overlap the sensor transmitter, the first over-tape bandage and the skin, thus further securing the sensor and the transmitter in place as well as providing a protective cover for the sensor assembly and the skin surface.

Such conventional over-tape adhesive bandages are used to provide a protective and stabilizing cover for the sensor assembly on the skin. However, application of the conventional adhesive bandages available requires several, complicated steps, making their use difficult and cumbersome for subjects and medical personnel. For example, the application procedures can include the stepwise removal of up to four or more adhesive backing sections from sections of two or more separate bandages in order to apply them to the skin and sensor assembly. The total amount of steps to adhere the sensor to skin can include six or more overall steps.

Such procedures require the repeated use of the user's fingers to peel away the several adhesive backing sections, substantially increasing the risk that the user's fingers will touch the adhesive surface of the bandage material that is applied to the sensor assembly and the patient's skin, increasing the risk of introducing contamination to the site of sensor portion insertion. Moreover, contacting the adhesive with the fingers can decrease the adhesive strength of the adhesive tape. For example, finger contact can cause contamination via the adherence of oils and dead skin cells to the adhesive, thus weakening the adherence of the adhesive tape to skin. This loss of adhesion can lead to a less stable sensor attachment, causing loosening of the sensor, which can cause bad sensor readings. This separation can cause inaccurate readings and subsequent mistreatments or even the sensor falling off the skin entirely. If the sensor detaches, then the user may need to purchase and apply a new sensor, which can lead to a potentially dangerous period of time without a sensor if a new one is not immediately available.

If that wasn't bad enough, the attachment of multiple strips of overlapping adhesive bandage to the skin reduces the breathability of the total adhesive assembly. Also, using multiple overlapping strips of adhesive bandage to secure a sensor to skin reduces the flexibility of the bandage material, which makes the total adhesive assembly stiff and uncomfortable. Binding a subcutaneous sensor to skin using an adhesive bandage is uncomfortable enough, especially for long term use. Having to use an adhesive assembly that is stiff and unbreathable just makes it more uncomfortable and can result in the adhesive assembly needing to be changed and moved to different sites on the skin frequently. The need for frequent changing and moving can also cause conventional bandage materials to fray or lift up from the skin soon after application, which can bother the wearer and have a visually displeasing appearance. Oh wait, it gets worse!

Sooner or later, most adhesive bandages need to be removed. Reversing the application procedure can include the stepwise removal of up to four or more adhesive strips from the skin. This can be significantly painful, and additionally so for bandages with stiff plastic or woven materials with limited flexibility. Many people debate whether it hurts more to remove an adhesive bandage quickly or slowly. Few people would debate that is hurts more to stepwise remove 4-7 adhesive bandages than it hurts to remove 1 or 2 adhesive bandages.

It has been discovered that it is possible to design an adhesive bandage that can be applied with as little as two adhesive steps, resulting in an adhesive bandage with as little as two minimally overlapping adhesive portions. In more detail, the adhesive bandage of the present disclosure can provide a benefit of the use of a single adhesive bandage for adhering a sensor to the skin of a patient in need thereof. In an embodiment, an adhesive bandage as disclosed herein can provide separate bandage panels, which can include a port front panel and a non-port front panel. The port front panel can include a port opening to allow application of the port front layer to the skin and over any adhesive portion included with the sensor assembly, while allowing the sensor port to extend through or remain accessible through the port opening above the surface of the skin. The non-port front panel can be applied over the skin and over an overlapping portion of the port front panel. The non-port front panel can also be applied to overlap and secure a sensor transmitter in place, wherein the sensor transmitter is connected to the sensor port.

It has been discovered that: by designing the front slit in the front layer and the back slit in the backing layer to be an overhang distance apart, an overhang is formed from the material of the front layer or the backing layer. The front slit allows an area of adhesive of the port front portion to be exposed and adhered to the skin and sensor surface while aligning the port opening with the sensor port, without touching the adhesive with the fingers. Once the port front portion is adhered to the skin and sensor surface, the port back panel can be removed from the overhang to expose an area of adhesive of the non-port portion, without touching the adhesive; then the overhang can be aligned over the port front portion and used to adhere the front non-port portion into contact with the port front portion and skin. The non-port back panel can then be removed while simultaneously adhering the remainder of the non-port front portion, using the already adhered overhang for stability, to complete the adhesive assembly without touching the non-port portion adhesive with the fingers. Adherence of the non-port portion can be performed by a user with one hand, which can be of importance to users applying a sensor to one arm.

One benefit of an embodiment of the adhesive bandage can be that the front layer includes a non-woven material. The woven layers of conventional adhesive bandages can suffer from fraying, which can lead to an undesirable appearance and accidental delamination as the frays catch on objects. The woven layers of conventional adhesive bandages also prevent or reduce the ability of the user to stretch the adhesive bandage during application, use, and removal of the adhesive bandage. In contrast, the non-woven material of the adhesive bandage disclosed herein can benefit from their ability to stretch in all or multiple directions during application, use, and removal of the adhesive bandage. This stretchable property can allow for the adhesive bandage to thin and lift from the skin in a non-abrasive way instead of pulling at the skin like conventional thicker or rigid bandages. In an embodiment of the adhesive bandage, the front layer excludes a woven material.

Another benefit can low manufacturing costs of the adhesive bandage disclosed herein. For example, adhesive bandages can be printed from a single manufactured unit that includes a front layer adhered to at least a portion of the lower surface of the front layer and a backing layer attached to the adhesive layer. This single manufactured unit can be custom ordered from commercial vendors. Then the process for making the adhesive bandage can include cutting at least one of the front layer and the backing layer of the pre-bandage material to form the adhesive bandage disclosed herein. This simplicity of manufacturing can greatly reduce costs by allowing for large volume, continuous high-throughput manufacturing of the adhesive bandage.

In an embodiment, the bandage panels can be formed of a front layer and an adhesive layer adhered to at least a portion of a lower surface of the front layer, the front layer and the adhesive layer being divided by at least one front slit to allow separation of the bandage panels. A backing layer adhered to the adhesive layer can include at least one back slit, where each back slit extends through the backing layer only and is located at an overhang distance from the front slit dividing the bandage panels.

A benefit to embodied methods of using the adhesive bandage herein can include the adhering of a subcutaneous sensor to the skin of a patient in an efficient and hygienic two-step application procedure. In embodiments herein, a port front bandage panel can be first adhered into place, followed by a second adhering of a non-port front panel. In such embodiments, the backing layer of the bandage panels can be delaminated to allow the bandage panels to be applied over an attached sensor and the skin surface, without the need of a user to touch the adhesive layer surface of the bandage panels, and without the need to touch any edges of the bandage backing layer with the fingers. The risk of touching and contaminating the surface of the adhesive layer during application of the bandage is thus greatly reduced. A benefit of embodied methods herein can thus include providing an improved efficient taping procedure with a lowered risk of contamination during application of a sensor to the skin of a patient. A further benefit of the adhesive bandage and method of applying the adhesive bandage can include an assembly with minimal or control overlap of the adhesive bandage layers.

### Front Layers of Various Embodiments

In various embodiments, the adhesive bandage includes a front layer. In an embodiment, the front layer includes a nonwoven material having an upper surface, a lower surface, and an outer edge. In an embodiment, the front layer has a length of from about 7.5 cm to about 15.2 cm, and a width of from about 7.5 cm to about 12.7 cm. In an embodiment, the front layer has a length of from about 7.0 cm to about 14.7 cm. In an embodiment, the front layer has a width of from about 7.0 cm to about 12.2 cm. If the front layer has a length of less than 7.5 cm, then the adhesive bandage may not adequately cover the sensor and adhere to enough surrounding skin to form an effective bond. If the front layer has a length of more than 15.2 cm, then the patient or subject may find the adhesive bandage too uncomfortable to wear for long durations. If the front layer has a width of less than 7.0 cm, then the adhesive bandage may not adequately cover the sensor and adhere to enough surrounding skin to form an effective bond. If the front layer has a width of more than 12.7 cm, then the patient or subject may find the adhesive bandage too uncomfortable to wear for long durations. In an embodiment, the front layer has a thickness of from about 2 micrometers to about 90 micrometers. In an embodiment, the front layer has a thickness of from about 20 micrometers to about 80 micrometers. In an embodiment, the front layer has a thickness of from about 40 micrometers to about 60 micrometers.

In various embodiments, the front layer is formed from one or more nonwoven materials suitable for use in an adhesive tape bandage. In an embodiment, the front layer is formed from one or more breathable materials suitable for use in an adhesive tape bandage for attachment to skin. In an embodiment, the nonwoven material includes polyurethane, polyvinylchloride, polyethylene, polyester, rayon, synthetic rubber, an elastic fabric, or combinations thereof. In some embodiments, the front layer has a circular shape or an oval shape.

In some embodiments, the adhesive bandage includes a display layer. In an embodiment, the display layer includes an ink, a dye, a pigment, or a combination thereof.

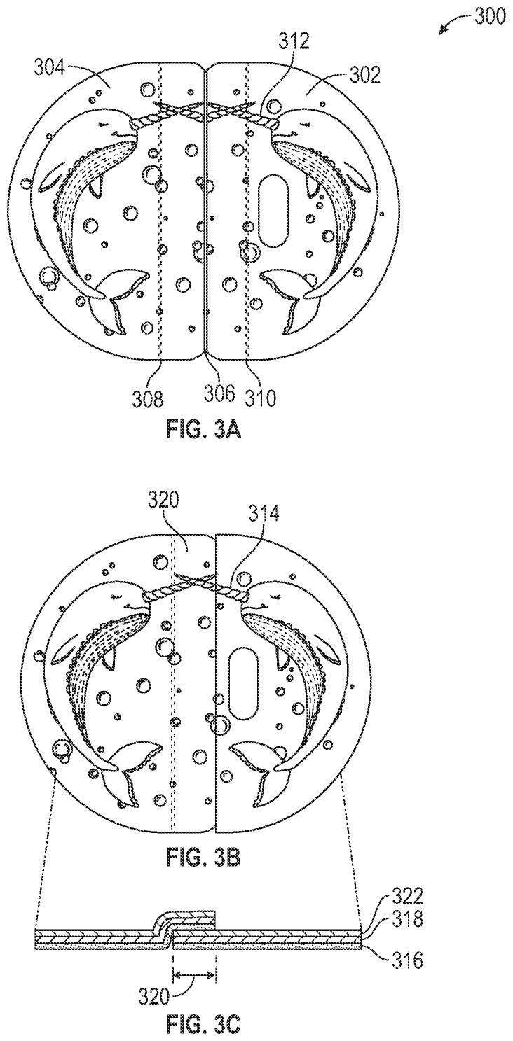

In such an embodiment, the front layer of an adhesive bandage can be printed with a pattern or design that is decorative and functional. Referring to FIG. 3A, an embodiment of the adhesive bandage 300, as disclosed herein, can having a port front panel 302 and a non-port front panel 304, wherein the port front panel and non-port front panel are separated by a front slit 306. In an embodiment, referring to FIG. 3C, the port front panel and non-port front panel include a front layer 318 in contact with an adhesive layer 316. In an embodiment, the port front panel and non-port front panel are at least partially coated with an ink layer 322 on the side of the front layer opposite of the adhesive layer. In an embodiment, referring to FIG. 3A, the ink layer can include an unresolved puzzle pattern or image 312, wherein the unresolved puzzle pattern is configured to be resolved into a resolved pattern 314 in FIG. 3B if the at least two separate bandage panels are overlapped by about the first overhang distance, or if applicable, a first and second overhang distance, to form an overlap region 320 as shown in FIG. 3B (top view) and FIG. 3C (side view). In such an embodiment, the design of the ink layer can guide the user in forming the proper amount of overlap.

### Adhesive Layers of Various Embodiments

In various embodiments, the adhesive bandage includes an adhesive layer adhered to at least a portion of the lower surface of the front layer. In an embodiment, the front layer and the adhesive layer have a combined thickness that is suitable for use in an adhesive bandage for attachment to skin. In an embodiment, the front layer and the adhesive layer have a combined thickness of from about 50 micrometers to about 190 micrometers. In an embodiment, the front layer and the adhesive layer have a combined thickness of from about 70 micrometers to about 130 micrometers. In an embodiment, the front layer and the adhesive layer have a combined thickness of from about 90 micrometers to about 110 micrometers. If the combined thickness of the front and adhesive layer exceeds 190 micrometers, then the adhesive bandage becomes impractically uncomfortable and loses breathability. If the combined thickness of the front and adhesive layer falls below 50 micrometers, then the adhesive bandage can become impractically weak and difficult to manipulate. In an embodiment, the adhesive layer does not extend to be coterminous with the outer edge of the front layer. In an embodiment, the adhesive layer does not extend to be coterminous with the outer edge and/or the inner edge of the front layer. In an embodiment, the adhesive layer extends to within from about 1 mm to about 2 mm of the outer edge and/or the inner edge of the front layer. If the adhesive layer extends to less than about 1 mm to the outer or inner edge, then the adhesive bandage can become difficult to remove. If the adhesive layer extends to more than 2 mm from the outer or inner edge, then the adhesive hold can be too weak to secure the sensor in place for prolonged use.

In an embodiment, the adhesive layer is formed from a tacky adhesive material. In an embodiment, the tacky adhesive material includes an acrylate resin, a methacrylate resin, an epoxy resin, and combinations thereof

### Backing Layers of Various Embodiments

In various embodiments, the adhesive bandage includes a backing layer attached to the adhesive layer. In an embodiment, the backing layer has a thickness of from about 50 micrometers to about 150 micrometers, including from about 80 to 120 micrometers, including from about 85 to about 115 micrometers. In an embodiment, the backing layer has a thickness of from about 3 micrometers to about 5 micrometers. In various embodiments, the backing layer is removable from the adhesive layer, and formed from one or more materials known to weakly adhere to tacky adhesives and suitable for use in adhesive bandage. In an embodiment, the backing layer is formed from a nonwoven elastic polymer material, a flexible plastic, a wax coated paper material, a silicone treated paper material, or a combination thereof.

### Bandage Panels of Various Embodiments

In various embodiments of an adhesive bandage, the front layer and the adhesive layer include at least two separate bandage panels, wherein the at least two separate bandage panels are divided by at least one front slit. In an embodiment, the at least two separate bandage panels include a port front panel and a non-port front panel. In such embodiments, the backing layer includes at least two backing panels, including a port back panel and a non-port back panel. In some embodiments, the at least two backing panels are separated by at least one back slit, wherein the at least one back slit is a first overhang distance from the front slit. In an embodiment, the first overhang distance of the at least one back slit is from about 0.5 cm to about 1.8 cm. In an embodiment, the first overhang distance of the at least one back slit is from about 0.7 cm to about 1.6 cm. In an embodiment, the first overhang distance of the at least one back slit is from about 0.9 cm to about 1.4 cm.

In an embodiment, the port front panel includes a port opening extending through at least the front layer. In an embodiment, the port opening has a length of from about 1.7 cm to about 3.8 cm, and a width of from about 0.7 cm to about 1.8 cm. In an embodiment, the port opening has a length of from about 1.5 cm to about 3.6 cm, and a width of from about 0.5 cm to about 1.6 cm. In an embodiment, the port opening has a circular shape or an oval shape. In an embodiment, the port opening has an inner edge, wherein the adhesive layer does not extend to be coterminous with the inner edge of the port opening.

In an embodiment of the adhesive bandage, the front layer and the adhesive layer include a port front panel and a non-port front panel divided by a front slit, and the backing layer includes a port back panel and a non-port back panel separated by a back slit, wherein the back slit is at an overhang distance from the front slit. In an embodiment, the overhang distance is positive and non-zero. In such an embodiment, the overhang distance is from about 0.5 cm to about 1.8 cm. In such an embodiment, the port front panel includes a port opening extending through at least the front layer.

In another embodiment of the adhesive bandage, the front layer and the adhesive layer include a port front panel and a non-port front panel divided by a front slit, and the backing layer includes a port back panel and a non-port back panel separated by a first back slit at a first overhang distance from the front slit, and a second back slit at a second overhang distance from the front slit. In such an embodiment, the backing layer further includes a bridging back panel between the first back slit and the second back slit. In such an embodiment, the second overhang distance of the second back slit from the front slit is from about 0.5 cm to about 1.8 cm. In an embodiment, the second overhang distance of the at least one back slit is from about 0.7 cm to about 1.6 cm. In an embodiment, the second overhang distance of the at least one back slit is from about 0.9 cm to about 1.4 cm.

In various embodiments of an adhesive bandage, the front layer and the adhesive layer include at least two separate bandage panels, wherein the at least two separate bandage panels are divided by at least one front slit, including from 1, 2, or 3 or more front slits. In an embodiment, the at least two separate bandage panels include at least one port front panel. A “port front panel” as used herein refers to a portion of the front layer having at least one opening having an area of at least one square centimeter. A “non-port front panel” as used herein refers to a portion of the front layer excluding an opening having an area of at least one square centimeter. One benefit to an adhesive bandage having a front layer and adhesive layer divided by at least one front slit, forming two or more port panels can be allowing for more than one sensor port to be accessed. Another benefit to an adhesive bandage having a front layer and adhesive layer divided by at least one front slit, for example, forming two port panels, can be that users can adhere either port panel to skin and the sensor first and then adhere the remaining port panel to skin and the first adhered port panel. This design would alleviate the need to determine which side is the port panel and apply it first because either side can be used first.

In an embodiment, the backing layer includes at least two backing panels, including at least a port backing panel and a non-porting back panel; or at least two port backing panels. A port backing panel refers to a portion of the backing layer that is adhered, directly or indirectly, to a port front panel. A non-port backing panel refers to a portion of the backing layer that is adhered, directly or indirectly, to a front non-port panel. In some embodiments, the at least two backing panels are separated by at least one back slit, including 2, 3, or 4 backing slits.

Generally, the purpose of the backing layer is merely to prevent accidental or premature adhesion of the adhesive bandage to a surface other than the site where it is intended to attach the sensor. However, it has been discovered that if the backing slit of the backing layer and the front slit of the front layer are an overhang distance apart, then an overhang of backing layer or front layer and adhesive is formed after the first front layer portion, usually the port front layer, is adhered to the sensor and/or skin. It was discovered that the overhanging portion of the front layer or the backing layer could be used to manipulate and adhere each successive portion of front layer portion. In an embodiment, the overhang distance is from about 0.5 cm to about 1.8 cm. If the overhang distance falls below 0.5 cm, then the ability to adhere the second portion of the adhesive bandage to the skin and sensor without having to touch the adhesive layer is lost. If the overhang layer exceeds 1.8 cm, then the overhang formed would be difficult to manipulate without having to touch the adhesive layer, and the overlap of the at least two front portions would result in a thick overlap region, rendering the adhesive bandage assembly uncomfortable.

A benefit of the overhang portion can include allowing for the adhesive/front panel component to be partially adhered. This allows for two things. First, the user is able to align the hole or port opening over the sensor assembly without touching the adhesive material. Second, when a portion of it is secure it is easier to remove the rest of the backing without touching the material, and with only one hand. This benefit can be especially important for users who apply the device to their arm.

In an embodiment, the overall shape of the front layer, including the inner edge, outer edge, and front portions is not limited. In an embodiment, the at least one front slit may have a shape that is straight, curved, or have a pattern, such as a scalloped and zig-zag design. In an embodiment, the pattern of the front slit can be regular or irregular, provided that the overhang is present at one or two points of the front slit. If front slit and the back slit are not straight and parallel, then the overhang distance is measured from the longest distance between the front slit and back slit. In an embodiment, the front slit and the back slit can be parallel, substantially parallel, or at any intersecting angle relative to one another, including between 0 to 45 degrees.

### Methods of Use of Embodiments Herein

Various embodiments herein present methods of adhering a sensor to a skin of a subject in need thereof. Such methods may be practiced using various embodiments of the adhesive bandages disclosed herein. In an embodied method, the adhering of a sensor to a skin of a subject in need thereof is accomplished by steps including: providing an adhesive bandage according to embodiments herein, adhering at least a portion of the port front layer into contact with an adhesive portion of the sensor and at least a portion of the port front layer into contact with the skin of the subject; and adhering at least a portion of the non-port (or second port front) layer into contact with the upper surface of the port front layer and onto the skin of the subject. In an embodiment, the method includes connecting a sensor transmitter through the port opening to the sensor. In an embodiment, the sensor transmitter is connected to the sensor before adhering the port front layer into contact with the sensor and the skin of the subject. In another embodiment, the method includes connecting a sensor transmitter through the port opening to the sensor during adhering the port front layer into contact with the sensor and the skin of the subject. In another embodiment, the method includes connecting a sensor transmitter through the port opening to the sensor after adhering the port front layer into contact with the sensor and the skin of the subject.

In embodiments of the methods, the sensor can include a sensor portion, an adhesive portion, and an exterior portion. In such embodiments, a sensor portion can include a sensor probe or wire that is inserted under the surface of the skin. In such embodiments, an adhesive portion of the sensor can include an adhesive pad that can be included as part of the sensor to aid the initial attachment of the sensor to the skin prior to the embodiments of the present method. In such embodiments, an exterior portion can include a sensor port that extends above the surface of the skin.

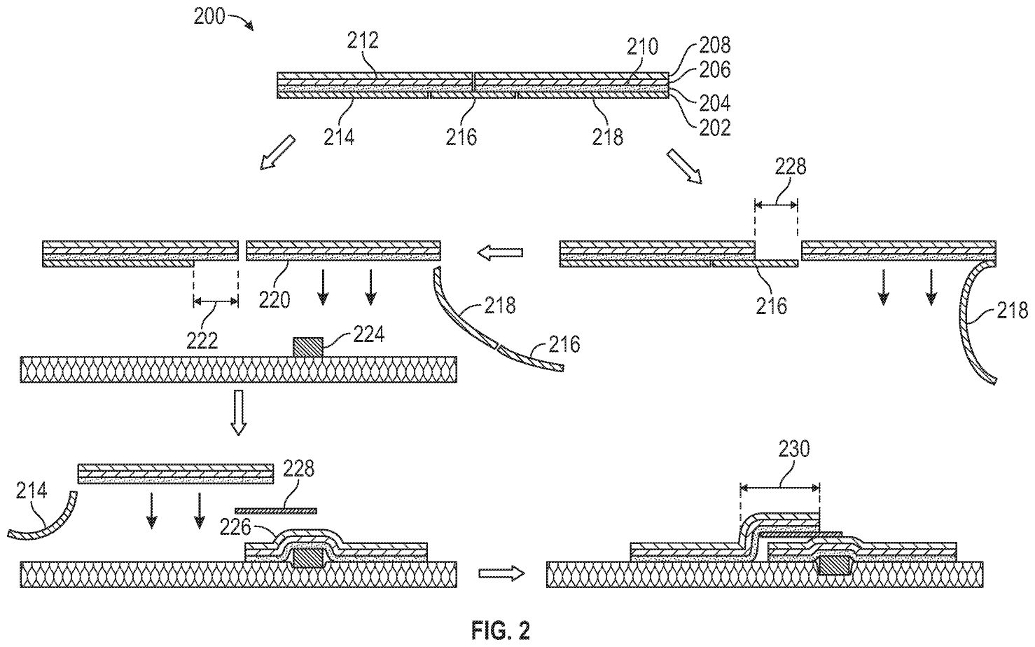

Embodiments of methods of adhering an embodiment of an adhesive bandage 200, as disclosed herein, are shown in FIG. 2. Referring to FIG. 2 (left side), the adhesive bandage can include a backing layer 202, an adhesive layer 204, a front layer 206, and an ink layer 208. In an embodiment of the methods herein, a port front panel 210 can be first adhered into place, followed by a second adhering of a non-port front panel 212 or second port front panel. In such an embodiment, an adhesive bandage, according to embodiments herein, can be utilized by a user by first pushing against the bandage panels to delaminate a portion of the port front panel 210 from the port back panel 218 and the bridging back panel 216 along the front slit, exposing the adhesive layer of the port front panel 220 and forming an adhesive overhang 222 on the non-port front panel. The user can then adhere the adhesive layer of the port front panel 220 directly to the skin and over any sensor adhesive portion 224 without touching the port front panel adhesive layer, allowing the sensor port to extend through the port opening.

In a next step, the user can attach a transmitter 228 over the port front portion, through a port opening to the sensor port. Then, the user can then adhere the adhesive overhang 222 of the non-port panel 212 to the skin and the top of the port panel portion 226, adhering the transmitter into place. Then, by applying pressure to the non-port panel portion, the non-port back panel 214 can be delaminated. When a sufficient portion of the non-port back panel has been delaminated, the non-port back panel can be adhered to the skin and the top of the port panel portion 226 without touching the adhesive surface to form an adhesive bandage secured sensor, wherein the adhesive bandage secured sensor has an overlap region 230 where the non-port front panel overlaps the port front panel.

One benefit of this method can include that the port front panel allows the inserted “sensor assembly” to remain stable while the patient or user clips in their transmitter, then the non-port front panel can be adhered to secure the transmitter so it does not loosen or disconnect during everyday use. Also, the appearance of the non-port front panel touching and overlapping the port front panel can form a seamless pattern that is visually pleasing and very secure.

In another embodiment of the methods herein, referring to FIG. 2 (right side), an adhesive bandage, according to embodiments herein, can be utilized by a user by first pushing against the bandage panels to delaminate a portion of the port front panel adhesive from the bridging back panel 216 along the front slit, exposing the adhesive layer of the port front panel 220 and forming a non-adhesive overhang 228, wherein the bridging back panel 216 remains attached the non-port front panel. The user can then attach the adhesive layer of the port front panel 220 directly to the skin without touching the adhesive surface or any edges of the backing layer.

In a next step, the user can manually remove the non-adhesive overhang portion 228 of the bridging back panel 216 by peeling the bridging back panel away from the non-port panel along a second back slit, again without touching the adhesive layer, to form an adhesive overhang 222.

In a next step, the user can attach a transmitter 228 over the port front portion, through port opening to the sensor port. Then, the user can then adhere the adhesive overhang 222 of the non-port panel 212 to the skin and the top of the port panel portion 224, adhering the transmitter into place. Then, by applying pressure to the non-port panel portion, the non-port back panel 214 can be delaminated. When a sufficient portion of the non-port back panel has been delaminated, the non-port back panel can be adhered to the skin and the top of the port panel portion 226 without touching the adhesive surface to form an adhesive bandage secured sensor 230, wherein the adhesive bandage secured sensor has an overlap region 230 where the non-port front panel overlaps the port front panel.

### Methods of Manufacture of Embodiments Herein

Various embodiments herein present methods of manufacturing an adhesive bandage. An embodiment of the methods provides a pre-bandage material. In an embodiment, the pre-bandage material includes a front layer including a nonwoven material and having an upper surface, a lower surface and an outer edge; an adhesive layer adhered to at least a portion of the lower surface of the front layer, and a backing layer attached to the adhesive layer. Embodiments of the methods provide cutting at least one of the front layer and the backing layer of the pre-bandage material to form an adhesive bandage according to various embodiments herein. In an embodiment, the method includes cutting a front slit in the front layer and a back slit in the back layer with a first overhang distance of from about 0.5 cm to about 1.8 cm.

### Examples

A pre-bandage material 3M™ 9907HTW single-sided elastic nonwoven fabric medical tape, 60# liner, or 3M™ 1530 microporous rayon nonwoven fabric medical tape with liner, was used to manufacture an adhesive bandage according to an embodiment of the specifications shown in FIG. 1. The manufactured adhesive bandage is suitable for use in over-tape procedures in attaching subcutaneous glucose monitors to the skin of patients, including but not limited to the Medtronic Guardian, Enlite, Minimed, and MiniMed™ 670G subcutaneous glucose monitors. The adhesive was first printed to display patterns that overlap in a meaningful way as shown in FIGS. 1-3 and then was cut using a variation of die-cuts and kiss-cuts to create shapes shown in the Figures.

What is claimed is:

1. An adhesive bandage comprising:
a front layer, wherein the front layer includes a nonwoven material and the front layer has an upper surface, a lower surface, and an outer edge;
an adhesive layer adhered to at least a portion of the lower surface of the front layer; and
a backing layer attached to the adhesive layer;
wherein the front layer and the adhesive layer include at least two separate bandage panels, wherein the at least two separate bandage panels are divided by at least one front slit, and the at least two separate bandage panels include a port front panel and a non-port front panel,
wherein the port front panel includes a port opening extending through at least the front layer;
wherein the backing layer includes at least two backing panels, wherein the at least two backing panels include a port back panel and a non-port back panel, wherein the at least two backing panels are separated by at least one back slit; and
wherein the at least one back slit is a first overhang distance from the front slit.

2. The adhesive bandage of claim 1, wherein the front layer has a width of from about 7.5 cm to about 15.2 cm, and a length of from about 7.5 cm to about 12.7 cm.

3. The adhesive bandage of claim 1, wherein the port opening has a length of from about 1.7 cm to about 3.8 cm, and a width of from about 0.7 cm to about 1.8 cm.

4. The adhesive bandage of claim 1, wherein the front layer has a thickness of from about 2 micrometers to about 90 micrometers.

5. The adhesive bandage of claim 1, wherein the front layer and the adhesive layer have a combined thickness of from about 50 micrometers to about 150 micrometers.

6. The adhesive bandage of claim 1, wherein the backing layer has a thickness of from about 50 micrometers to about 150 micrometers.

7. The adhesive bandage of claim 1, wherein the first overhang distance of the at least one back slit from the front slit is from about 0.5 cm to about 1.8 cm.

8. The adhesive bandage of claim 1, wherein the nonwoven material includes polyurethane, polyvinylchloride, polyethylene, polyester, rayon, synthetic rubber, an elastic fabric, or combinations thereof.

9. The adhesive bandage of claim 1, wherein the adhesive layer is formed from a tacky adhesive material.

10. The adhesive bandage of claim 9, wherein the tacky adhesive material includes an acrylate resin, a methacrylate resin, an epoxy resin, and combinations thereof.

11. The adhesive bandage of claim 1, wherein the backing layer is formed from a nonwoven elastic polymer material, a flexible plastic, a wax coated paper material, a silicone treated paper material, or a combination thereof.

12. The adhesive bandage of claim 1, wherein the port back panel and the non-port back panel are separated by a second back slit, wherein the second back slit is a second overhang distance from the front slit; and wherein the backing layer further comprises a bridging back panel between the first back slit and the second back slit.

13. The adhesive bandage of claim 15, wherein the second overhang distance of the second back slit from the front slit is from about 0.5 cm to about 1.8 cm.

14. The adhesive bandage of claim 1, further comprising a display layer, wherein the display layer includes an ink, a dye, a pigment, or a combination thereof.

15. The adhesive bandage of claim 14, wherein the upper surface of the front layer is printed with a decorative design, wherein the display layer includes an unresolved puzzle pattern, and the unresolved puzzle pattern is configured to be resolved into a resolved pattern if the at least two separate bandage panels are overlapped by about the first overhang distance.

16. The adhesive bandage of claim 1, wherein the front layer has a circular or an oval shape, or the port opening has a circular or an oval shape.

17. The adhesive bandage of claim 1, wherein the port opening has an inner edge, wherein the adhesive layer does not extend to be coterminous with the outer edge of the front layer, or wherein the adhesive layer does not extend to be coterminous with the inner edge of the port opening.

18. A method of adhering a sensor to a skin of a subject in need thereof, comprising:
placing the sensor into contact with the skin of the subject;
providing the adhesive bandage of claim 1;
adhering at least a portion of the port front layer into contact with an adhesive portion of the sensor and at least a portion of the port front layer into contact with the skin of the subject; and
adhering at least a portion of the non-port front layer into contact with the upper surface of the port front layer and onto the skin of the subject.

19. The method of claim 18, further comprising:
before, during, or after adhering the port front layer into contact with the sensor and the skin of the subject,
connecting a sensor transmitter through the port opening to the sensor; or
wherein the sensor includes a sensor portion, the adhesive portion, and an exterior portion.

20. (canceled)

21. A method of manufacturing an adhesive bandage comprising:
providing a pre-bandage material, wherein pre-bandage material includes a front layer, wherein the front later includes a nonwoven material and the front layer has an upper surface, a lower surface, and an outer edge;
an adhesive layer adhered to at least a portion of the lower surface of the front layer;
a backing layer attached to the adhesive layer; and
cutting at least one of the front layer and the backing layer of the pre-bandage material to form the adhesive bandage of claim 1.
